(19) United States
(12) Patent Application Publication
(10) Pub. No.: US 2011/0020829 A1
(43) Pub. Date: Jan. 27, 2011

(54) RAPID ASSAY FOR DETECTING ATAXIA-TELANGIECTASIA HOMOZYGOTES AND HETEROZYGOTES

(75) Inventors: Richard Gatti, Los Angeles, CA (US); Shareef A. Nahas, Los Angeles, CA (US); Anthony W. Butch, Los Angeles, CA (US)

Correspondence Address: KNOBBE MARTENS OLSON & BEAR LLP, 2040 MAIN STREET, FOURTEENTH FLOOR, IRVINE, CA, 92614, US

(73) Assignee: The Regents of the University of California, Oakland, CA (US)

(21) Appl. No.: 12/921,758

(22) PCT Filed: Mar. 13, 2009
(86) PCT No.: PCT/US09/37201

§ 371 Date: Sep. 9, 2010

Related U.S. Application Data
(60) Provisional application No. 61/036,829, filed on Mar. 14, 2008.

(51) Int. Cl. C12Q 1/68

(52) U.S. Cl. 435/6

(57) ABSTRACT

The present disclosure relates to methods for performing an assay to identify ataxia-telangiectasia homozygotes or heterozygotes. Some embodiments include the use of a rapid flow cytometry-based ataxiatelangiectasia (ATM) kinase assay that measures ATM-dependent phosphorylation of SMC1 following DNA damage.

### CROSS-REFERENCE TO RELATED APPLICATIONS

This application claims the benefit from U.S. Provisional Patent Application 61/036,829 entitled RAPID ASSAY FOR ATAXIA-TELANGIECTASIA HOMOZYGOTES/HETEROZYGOTES filed on Mar. 14, 2008. The content of the aforementioned application is hereby incorporated by reference in its entirety.

### STATEMENT REGARDING FEDERALLY SPONSORED R&D

This invention was made with Government support by Grant Nos. NS052528 and AI067769, awarded by the National Institutes of Health. The Government may have certain rights in this invention.

### BACKGROUND OF THE INVENTION

1. Field of the Invention

The present invention relates to assays for identifying ataxia-telangiectasia homozygotes and heterozygotes, diagnosing ataxia-telangiectasia and/or cancer susceptibility in patients.

2. Description of the Related Art

Ataxia-telangeictasia (A-T) is a progressive neurodegenerative disorder of childhood onset, inherited in an autosomal recessive pattern. Patients are affected by a large range of symptoms including telangiectasia (dilation of blood vessels) on the eyes, face, and shoulders, ataxia (loss of balance), neurodegeneration, cerebellar degeneration, ocular telangiectasia, radiosensitivity, cancer predisposition, immunodeficiency, and premature aging. A-T cells display cell cycle checkpoint defects, chromosomal instability, and sensitivity to ionizing radiation.

The A-T gene, cloned by positional cloning (Savitsky et al. (1995) Hum. Mol. Genet. 4:2025-2032), encodes a 370 kDa protein kinase known as “ataxia-telangiectasia, mutated” (ATM) involved with the DNA double-stranded break response mechanism and initiation of DNA repair, which are events responsible for maintaining the genomic integrity of the cell. Activation of ATM has effects on multiple signal transduction pathways related to cell cycle checkpoints and DNA damage repair. Complete genomic sequence (184 kb) of the A-T gene, also known as the ATM gene, is disclosed at GenBank Accession No. U82828 (Platzer et al. (1997) Genome Res 7(6):592-605). ATM mRNA is disclosed at GenBank Accession No. U33841 (Savitsky et al. (1995) Hum. Mol. Genet. 4:2025-2032). Cloning, sequences, and organization of the A-T gene are disclosed, inter alia, in U.S. Pat. Nos. 6,265,158, 6,211,336 and 5,858,661 to Shiloh et al., and mutations in the A-T gene are disclosed in U.S. Pat. No. 5,955,279 to Gatti et al.

ATM is a serine/threonine kinase that belongs to a family of large kinases containing a C-terminal end homologous to the phosphatidylinositol 3-kinase domain. These proteins play a role in cell cycle checkpoint or DNA damage repair. Other members in this family include Rad 3, Mec1p, Mei-41, Rad 50, Tel1 and DNA-PK proteins. After DNA damage, ATM phosphorylates over 700 target proteins involved in cell-cycle checkpoints, apoptosis, nonsense-mediated decay, oxidative stress response, and DNA repair (Matsuoka et al. (2007) Science 316(5828):1160-1166). These processes involve proteins such as protein 53 (p53), check-point kinase (CHK2), Nijmegen breakage syndrome 1 (NBS1), structural maintenance of chromosomes 1 (SMC1), γ histone 2A variant (γ-H2AX), Fanconi anemia complementation group 2 (FANCD2), and breast cancer susceptibility (BRCA1) (Bakkenist et al. (2003) Nature 421(6922):499-506). Several groups of interacting proteins influence the crucial S phase checkpoint, such as the ATM/CHK2/Cdc25A, ATM/NBS1/SMC1, FANCD2-BRCA1, and RAD50/MRE11/NBS1 complexes (Yazdi et al. (2002) Dev 16(5):571-582; Kitagawa et al. (2004) Genes Dev 18(12):1423-1438).

A-T results only in individuals who are homozygous for the A-T gene mutation, but carriers of A-T (individuals who are heterozygous for the A-T gene mutation) often exhibit adverse health effects as well. In particular, carriers of A-T have increased susceptibility to various forms of cancer, particularly breast cancer, as well as coronary disease, compared to their homozygous normal counterparts. In studying the relationship between A-T and breast cancer, Waha et al. analyzed ATM transcripts and found low concentrations in breast carcinomas, intermediate levels in benign lesions and high levels in normal breast tissue, concluding that the ATM gene may contribute to the development and/or malignant progression of breast carcinomas (Waha et al. (1998) Int J Cancer 78(3):306-309). Djuzenova et al. examined cells from healthy donors, breast cancer patients, A-T heterozygotes and A-T homozygotes and concluded that the cells of individuals from both A-T groups exhibited increased sensitivity to DNA damage induced by x-irradiation (Djuzenova et al. (1999) Lab Invest 79(6):699-705). In a statistical study of patients, Broeks et al. reported a nine-fold increase in breast cancer risk among A-T heterozygotes (Broeks et al. (2000) Am J Hum Genet. 66(2):494-500). Furthermore, Geoffroy-Perez et al. reported a 3.6-fold increase in breast cancer risk among A-T heterozygotes (Geoffroy-Perez et. al. (2002) Int J Cancer 99(4):619-623). Numerous other investigations have examined the connection between A-T and breast cancer. See e.g., Yuille et al. (1998) Recent Results Cancer Res 154:156-173; Meyn (1999) Clin Genet. 55(5):289-304; Khanna (2000) J Natl Cancer Inst 92(10):795-802; Geoffroy-Perez et al. (2001) Int J Cancer 93(2):288-293. Some research also indicates an increased susceptibility to ischemic heart disease for A-T heterozygotes. See e.g., Su et al. (2000) Ann Intern Med 133(10):770-778; Swift et al. (1991) N Engl J Med 325(26):1831-1836. It is estimated that approximately 0.5% to 1% of the general population are carriers of A-T.

Presently, a diagnosis of A-T takes approximately 12 weeks and often is required on infants who are unable to provide more than a few milliliters of blood (Huo et al. (1994) Cancer Res 54(10):2544-2547; Sun et al. (2002) J Pediatr 140(6):724-731; Chun et al. (2003) Mol Genet Metab 80(4):437-443). The diagnostic protocol includes establishing a lymphoblastoid cell line (LCL) from whole blood, performing a colony survival assay (CSA) for radiosensitivity, and immunoblotting to determine the presence or absence of ATM protein, which is absent in >99% of A-T patients. Although the current diagnostic protocol is extremely sensitive, it is labor intensive and has a long turnaround time. The inventors recently developed a highly accurate immunoassay (ATM-ELISA) to identify ATM-deficient patients (Butch et al. (2004) Clin Chem 50(12):2302-2308; US Patent Publication No. 2004/0029198). The ATM-ELISA assay measures ATM protein concentrations directly from whole blood and confirms a diagnosis of A-T within 2 days on small numbers of peripheral blood mononuclear cells (PBMCs). The ATM-ELISA assay requires a purified ATM protein standard (Chun et al. (2004) Biochem Biophys Res Commun 322(1):74-81) and does not identify rare A-T patients with kinase-dead ATM protein. Furthermore, the variability of unbound ATM nuclear protein in fresh blood cells does not allow a reliable diagnosis of heterozygosity (Butch et al. (2004) Clin Chem 50:2302-2308). Because of the large size of the ATM gene, the cost of sequencing approximately 15,000 nt, the frequency of missed mutations (approximately 10%), and the limitations of sequence interpretation, direct ATM sequencing is not the recommended test of first choice for establishing a diagnosis; it is best reserved for confirmed A-T cases, in whom the consequences of specific mutations may influence both phenotype and future therapy (Lai et al. (2004) Proc Natl Acad Sci 101(44): 15676-15681).

Identifying heterozygosity in the absence of a prior affected family member is even more challenging. The goal in such cases is to establish whether a single ATM DNA change of consequence (i.e., a mutation) is present. ATM protein levels are usually 40-50% of normal in heterozygotes but cannot be reliably quantified by immunoblotting or ATM-ELISA from a single peripheral blood sample (Chun et al. (2003) Mol Genet Metab 80(4):437-443; Butch et al. (2004) Clin Chern 50(12):2302-2308). Radiosensitivity (CSA) testing of cell lines from known A-T heterozygotes using CSAs under hypoxic conditions is usually inconclusive, yielding scores in the normal or intermediate range (Paterson et al. (1985) New York: Liss, Kroc Found Series 19:73-87). Even the most rigorous efforts at heterozygote identification have never exceeded 80%-90% accuracy (Weeks et al. (1991) Radiat Res 128:90-99) and are not practicable for clinical testing.

Because isolation of purified ATM protein has been so difficult, assays which use ATM for diagnosing patients have been impractical or even impossible. There exists an unmet need in the art for a rapid and reliable assay for diagnosing A-T by identifying A-T homozygotes or heterozygotes based on ATM kinase function. Because of the link between A-T and cancer, particularly breast cancer, there also exists an unmet need for a method of diagnosing cancer susceptibility involving an assay which can detect and/or quantify functional ATM protein in a patient. Further, there exists an unmet need for an assay which can distinguish between individuals who are homozygous A-T (meaning homozygous for the mutated A-T gene), heterozygous carrier (meaning heterozygous with one mutated A-T gene and one normal A-T gene), and homozygous normal (meaning homozygous for the normal A-T gene). Since the health concerns of individuals in each of those three classes are unique, it would be advantageous to tailor patient counseling, further testing, and medical treatment in light of a patient's A-T genotype.

### SUMMARY OF THE INVENTION

In some embodiments, a method of detecting an ataxia-telangiectasia (A-T) gene mutation in a patient is provided, the method comprising the steps of: measuring the phosphorylation level of an ATM kinase target in a biological sample from the patient; contacting the biological sample with a DNA damage-inducing agent; measuring the phosphorylation level of the ATM kinase target in the biological sample after treatment with the DNA damage-inducing agent; and comparing the measured phosphorylation level before and after treatment with the DNA damage-inducing agent to determine the presence of an A-T gene mutation in the patient. The patient can be homozygous for the A-T gene mutation, homozygous normal with respect to the A-T gene mutation, or heterozygous for the A-T gene mutation. In preferred embodiments, the ATM kinase target is SMC1. Some preferred embodiments include use of ionizing radiation (IR) or bleomycin to generate DNA damage in a biological sample. Some preferred embodiments also include use of flow cytometry or immunoblot analysis to measure the phosphorylation level of the ATM kinase target. Preferred biological samples are peripheral mononuclear cells or lymphoblastoid cells.

In some embodiments, a method of screening for susceptibility of a disorder in a patient is provided, the method comprising the step of: measuring the phosphorylation level of an ATM kinase target in a biological sample from the patient; contacting the biological sample with a DNA damage-inducing agent; measuring the phosphorylation level of the ATM kinase target in the biological sample after treatment with the DNA damage-inducing agent; and comparing the measured phosphorylation level before and after treatment with the DNA damage-inducing agent to determine the susceptibility of a disorder in the patient.

In some embodiments, a kit for detecting an ataxia-telangiectasia (A-T) gene mutation in a patient, including a DNA damage-inducing agent, an antibody for detecting the phosphorylation level of an ATM kinase target and an instruction for contacting the DNA damage-inducing agent with a biological sample from the patient. Preferably the antibody is labeled with a fluorophore. In some embodiments, the kit further comprises a second antibody that binds to the antibody for detecting the phosphorylation level of the ATM kinase target. Preferably, the second antibody is labeled with a fluorophore.

### BRIEF DESCRIPTION OF THE DRAWINGS

FIG. 1 is an immunoblot to detect SMC1pSer966 in nuclear lysates using LCLs before (−) and after (+) 10 Gy IR.

FIGS. 2A-C are FC-pSMC1 histograms showing IR-induced ATM-dependent phosphorylation of SMC1 using LCLs.

FIG. 3 is a dot plot showing FC-pSMC1 data performed on LCLs from 7 healthy unknowns, 4 A-T heterozygotes, and 10 A-T homozygotes.

FIGS. 4A-D are FC-pSMC1 histograms showing IR-induced ATM-dependent phosphorylation of SMC1 using PBMCs.

FIG. 5 is a dot plot showing normalized FC-pSMC1 data performed on PBMCs from 16 healthy unknown, 10 A-T heterozygotes, and 10 A-T homozygotes.

FIGS. 6A-D are FC-pSMC1 histograms showing bleomycin-induced ATM-dependent phosphorylation of SMC1 using PBMCs.

FIG. 7 is a dot plot showing imprecision/variance of the FC-pSMC1 ATM kinase assay.

FIG. 8A is a dot plot showing FC-pSMC1 data performed on LCLs from a healthy unknown, 4 A-T heterozygotes (ATHET 1-4), 2 A-T (AT153LA and GRAT1), and a daily control; FIG. 8B is a bar graph showing ATM protein levels in nuclear lysates (100 μg) from the same LCLs used in FIG. 8A measured by ATM-ELISA.

FIGS. 9A-H are FC-pSMC1 histograms showing IR-induced ATM-dependent phosphorylation of SMC1 using LCLs for WT, A-T, and other genomic instability disorders.

FIG. 10 is an immunoblot of LCLs used for FC-pSMC1 assay in FIGS. 9A-D, developed with antibody to SMC1pSer966 for nuclear lysates of WT, A-T, Mrel1 and NBS cells after 10 Gy IR.

### DETAILED DESCRIPTION

Some embodiments relate to methods for detecting an ataxia-telangiectasia (A-T) gene mutation in a patient. The patient can be homozygous for the A-T gene mutation, homozygous normal with respect to the A-T gene, or heterozygous for the A-T gene mutation.

Some embodiments relate to methods for diagnosing a patient for ataxia-telangiectasia (A-T) and/or susceptibility to various conditions. There conditions can include cancer, particularly breast cancer, and heart disease. Some embodiments relate to the discovery that persons having an A-T gene mutation, including A-T heterozygotes, have an increased risk of developing some neurological disorders. Accordingly, susceptibility to these various conditions can be diagnosed by measuring the change in the phosphorylation level of an ATM kinase target in a patient's biological sample in response to a DNA damage-inducing agent. Diagnosis is generally performed in patients suspected of having or developing these conditions.

Preferably, the biological sample from the patient is blood. More preferably, the biological samples are peripheral blood mononuclear cells or lymphoblastoid cells. In some embodiments, cells are extracted from a patient's blood and the phosphorylation level of an ATM kinase target in nuclear cell lysate is determined by an assay. In the assay, the phosphorylation level of an ATM kinase target after the cells are treated with a DNA damage-inducing agent is measured and advantageously compared to the phosphorylation level of the ATM kinase target before the cells are treated with the DNA damage-inducing agent to determine the change in the phosphorylation level of the ATM kinase target in response to the DNA damage-inducing agent. The result of the assay are used to diagnose whether the patient is a homozygous A-T (meaning homozygous for the mutated A-T gene), a heterozygous carrier (meaning heterozygous with one mutated A-T gene and one normal A-T gene), or a homozygous normal (meaning homozygous for the normal A-T gene).

### Measuring the Phosphorylation Level of a Kinase Target

Techniques and systems to measure the phosphorylation level of a kinase target in a biological sample are well known by person skilled in the art. For example, immunostaining assays that utilize antibody-based staining methods can be applied to detect the presence and quantity of a phosphorylated form of a specific kinase target in a biological sample. Conventional immunostaining techniques include, but are not limited to, immunohistochemistry, immunoblotting, flow cytometry, enzyme-linked immunosorbent assay (ELISA), and immuno-electron microscopy. In some embodiments, the target protein is a phosphorylated form of the kinase target, and an antibody that can specifically recognize the target protein is used.

To detect the target protein, a detectable label can be used to label an antibody specific to the target protein or a secondary antibody that can specifically recognize an antibody specific to the target protein. The detectable label can be a reporter enzyme. When exposed to an appropriate substrate, this enzyme reacts in such a manner as to produce a chemical moiety which can be detected, for example, by colorimetric, pectrophotometric, chemiluminescent, fluorometric or visual means. Enzymes which can be used to detectably label the reagents useful in the present invention include, but are not limited to, horseradish peroxidase, alkaline phosphatase, glucose oxidase, β-galactosidase, ribonuclease, urease, catalase, malate dehydrogenase, staphylococcal nuclease, asparaginase, .DELTA.-5-steroid isomerase, yeast alcohol dehydrogenase, .alpha.-glycerophosphate dehydrogenase, triose phosphate isomerase, glucose-6-phosphate dehydrogenase, glucoamylase and acetylcholinesterase. For descriptions of EIA procedures, see Voller et al., J. Clin. Pathol. 31:507-520 (1978); Butler, Meth. Enzymol. 73:482-523 (1981); Maggio (ed.), Enzyme Immunoassay, CRC Press, Boca Raton, 1980; Butler, In: Structure of Antigens, Vol. 1 (Van Regenmortel, M., CRC Press, Boca Raton, 1992, pp. 209-259; Butler, In: van Oss et al. (eds), Immunochemistry, Marcel Dekker, Inc., New York, 1994, pp. 759-803; Butler (ed.), Immunochemistry of Solid-Phase Immunoassay, CRC Press, Boca Raton, 1991). Determination of the presence and quantity of the target protein can be carried out by colorimetry to measure the colored product produced by conversion of a chromogenic target by the enzyme. Determination may also be accomplished by visual comparison of the colored product of the enzymatic reaction in comparison with appropriate standards or controls.

In some embodiments, the detectable label may be a radiolabel, and the assay termed a radioimmunoassay (RIA), is well known in the art. See e.g., Yalow et al. (1959) Nature 184:1648; Work et al. Laboratory Techniques and Biochemistry in Molecular Biology, North Holland Publishing Company, NY, 1978, incorporated by reference herein. The radioisotope can be detected by a gamma counter, a scintillation counter or by autoradiography.

In some embodiments, the detectable label bound to the antibody reagents may be a fluorophore. When the fluorescently labeled antibody is exposed to light of a proper wave length, its presence can then be detected due to fluorescence of the fluorophore. Among the most commonly used fluorophores are fluorescein isothiocyanate (FITC), rhodamine, phycoerythrin, phycocyanin, allophycocyanin, o-phthaldehyde, sulforhodamine 101 acid chloride (Texas Red), fluorescamine or fluorescence-emitting metals such as 152 Eu or other lanthanides. These metals are attached to antibodies using metal chelators. In some embodiments, the fluorescently labeled probe is excited by light and the emission of the excitation is then detected by a fluorometer or a photosensor such as CCD camera equipped with appropriate emission filters

The specific antibodies useful for detecting the target protein can also be detectably labeled by coupling to a chemiluminescent compound. The presence of a chemiluminescent-tagged antibody is then determined by detecting the luminescence that arises during the course of a chemical reaction. Examples of useful chemiluminescent labeling compounds are luminol, isoluminol, theromatic acridinium ester, imidazole, acridinium salt and oxalate ester. Likewise, a bioluminescent compound such as a bioluminescent protein may be used to label antibody reagent. Binding is measured by detecting the luminescence. Useful bioluminescent compounds include luciferin, luciferase and aequorin.

In some embodiments, flow cytometry (FC)-based techniques and systems are provided to measure the activity of ATM kinase by measuring the phosphorylation level of an ATM kinase target. To detect the presence and/or quantity of a phosphorylated form of an ATM kinase target in the cells, the cells can be processed using techniques and systems that are well known by person skilled in the art, including Fix & Perm cell permeabilization kit (Caltag Laboratories; Invitrogen, Carlsbad, Calif.) and Optimized Fixation Kits for Surface and Intracellular Flow Cytometry (Imgenex, San Diego, Calif.). After permeabilization of a cell membrane, cells are stained with detectable label-bound antibodies that are specific for the phosphorylated form of the ATM kinase target. The phosphorylation level of the ATM kinase target is then measured using flow cytometry. For example, samples can be analyzed using a FACScaliber (BD Biosciences) with Cell Quest software, which plots geometric mean fluorescence intensity (GMFI) on the x axis using a log scale. GMFI peaks are converted to a linear scale for calculating % of daily control (% DC). The mean GMFI peak (linear scale) of untreated cells is subtracted from the GM peak FI of treated cells to yield the difference (δ GMFI), and the δ GMFIs for all samples are normalized against the δ GMFI of a healthy daily control (DC) and expressed as a proportion (% DC). In some embodiments, the preferred ATM kinase target is SMC1. The flow cytometry (FC)-based assay using SMC1 as the ATM kinase target to detect ATM kinase activity is termed as FC-pSMC1 assay.

Other techniques and systems for detecting a target protein in its cellular location that are well known by persons skilled in the art can also be used. For example, a “fixing and permeabilization” procedure can be performed for intracellular staining. In this procedure, cells can be first fixed to ensure stability of the target protein and then permeabilized prior to staining. Available “fixing and permeabilization” methods include, but are not limited to: (1) formaldehyde followed by detergent treatment to disrupt cell membrane; (2) formaldehyde followed by methanol; (3) methanol followed by detergent (e.g., Tween-20 or Triton-X); and (4) acetone fixation and permeabilization. For detection of secreted proteins, Brefadin A and other compounds are often used as a Golgi-Block to prevent protein proteins being released from the golgi.

In some embodiments, an immunoblotting assay, or alternatively termed a western blot assay, is used to measure the phosphorylation level of an ATM kinase target. Immunoblotting assays allow the detection of specific proteins from extracts made from cells or tissues, before or after any purification steps. In such assays, gel electrophoresis is used to separate native or denatured proteins in a biological sample before the proteins are transferred to a synthetic membrane (typically nitrocellulose or polyvinylidene fluoride (PVDF)). The membrane is then probed using antibodies specific to a phosphorylated form of the ATM kinase target.

### DNA Damage-Inducing Agents

As used herein, the term “DNA damage-inducing agent” includes any known DNA damage-inducing agent, including but is not limited to a topoisomerase inhibitor, DNA binding agent, anti-metabolite, ionizing radiation (IR), virus, hydrolysis or thermal disruption, restriction enzyme, or a combination of two or more of such known DNA damaging agents.

A topoisomerase inhibitor can be a topoisomerase I (Topo I) inhibitor, a topoisomerase II (Topo II) inhibitor, or a dual topoisomerase I and II inhibitor. Examples of preferred Topo I inhibitors include but are not limited to camptothecin, topotecan, irinotecan, belotecan, or an analogue or derivative thereof. Examples of preferred Topo II inhibitors include but are not limited to doxorubicin, etoposide phosphate, teniposide, sobuzoxane, or an analogue or derivative thereof.

DNA binding agents include but are not limited to DNA groove binding agents, e.g., DNA minor groove binding agents, DNA crosslinking agents, intercalating agents, and DNA adduct forming agents. A DNA minor groove binding agent can be an anthracycline antibiotic, mitomycin antibiotic, chromomycin A3, or an analogue or derivative thereof. DNA crosslinking agents include but are not limited to antineoplastic alkylating agents (e.g., cisplatin), methoxsalen, mitomycin antibiotic, or psoralen. Intercalating agents can be an anthraquinone compound, bleomycin, or an analogue or derivative thereof. DNA adduct forming agents include but are not limited to an enediyne antitumor antibiotic, platinum compound, carmustine, tamoxifen, psoralen, pyrazine diazohydroxide, or an analogue or derivative thereof.

Anti-metabolites include but are not limited to cytosine, arabinoside, floxuridine, fluorouracil, mercaptopurine, gemcitabine, and methotrexate (MTX).

The term “ionizing radiation” or “IR” as used herein includes, but is not limited to X-ray radiation, gamma-ray radiation, and ultraviolet light radiation.

### ATM Kinase Target

In response to DNA damage, ATM is activated, leading to a cascade of kinase reactions to phosphorylate over 700 target proteins involved in cell-cycle checkpoints, apoptosis, nonsense-mediated decay, oxidative stress response, and DNA damage repair (Matsuoka et al. (2007) Science 316(5828):1160-1166). Many target molecules of ATM kinase have been identified. As used herein, the term “ATM kinase target” includes any protein that is a substrate for ATM kinase, including but is not limited to, ataxia-telangiectasia mutated (ATM), protein 53 (p53 or TP53), check-point kinase (CHK2), Nijmegen breakage syndrome 1 (NBS1), structural maintenance of chromosomes 1 (SMC1), γ histone 2A variant (γ-H2AX), Fanconi anemia complementation group 2 (FANCD2), mediator of damage checkpoint 1 (MDC1), nuclear factor of kappa light polypeptide gene enhancer in B-cells inhibitor, alpha (NFKBIA), CtBP-interacting protein (CTIP), nibrin (NBN), telomeric repeat binding factor (TERF1), RAD9, RAD17, DNA cross-link repair 1C (DCLRE1C), Artemis, stress responsive activator of p300 (Strap), E2F transcription factor 1 (E2F1), Oligonucleotide/oligosaccharide-binding fold-containing protein 2B (OBFC2B), Che1, and breast cancer susceptibility (BRCA1) (See e.g., Bakkenist et al. (2003) Nature 421(6922):499-506; Yan et al. (2008) Cancer Lett 271(2):179-190; Zhang et al. (2004) Mol Cell Biol 24(20):9207-9220; Adams et al. (2008) EMBO reports 9(12):1222-1129; Bruno et al. (2006) Cancer Cell 10(6):473-486).

The phosphorylation of SMC1 protein by ATM kinase, following ATM kinase recruitment and activation by NBS1 and BRCA1 to DNA double strand break (DSBs) damage sites, is thought to play an important role in the rapid cellular response to radiation damage. SMC1 protein is directly phosphorylated by ATM kinase on serines 957 and 966 in response to DNA damage (Bakkenist et al. (2003) Nature. 421:499-506; Yazdi et al. (2002) Genes Dev 6(5):571-582; Kitagawa et al. (2004) Genes Dev 18(12):1423-1438). Accordingly, SMC1 can be used as an ATM-dependent target to detecting ATM kinase activity.

Antibodies specific to ATM kinase targets and phosphorylated forms of these ATM kinase targets include, but are not limited to: rabbit anti-ATMpSer1981 (GenScript, Piscataway, N.J.), rabbit anti-SMC1pSer966 (Novus, Littleton, Colo.), rabbit anti-SMC1pSer957 (Abeam, Cambridge, Mass.), rabbit anti-H2AXpSer139 (Abeam, Cambridge, Mass.), rabbit anti-FANCD 2pSer222 (Abeam, Cambridge, Mass.), rabbit anti-p53pSer15 (Abeam, Cambridge, Mass.), rabbit anti-NBS1pSer343 (Abeam, Cambridge, Mass.), rabbit anti-BRCA1pSer1423 (Abeam, Cambridge, Mass.), rabbit anti-BRCApSer1387 (Abeam, Cambridge, Mass.).

In some embodiments, a kit or reagent system useful for practicing the methods described herein is provided. Such a kit will generally contain a reagent combination comprising the elements required to conduct an assay according to the disclosed methods and an instruction for practicing the disclosed methods. The reagent system can be presented in a commercially packaged form, as a composition or admixture (where the compatibility of the reagents allow), in a test device configuration, or more typically as a test kit. A test kit is typically a packaged combination of one or more containers, devices, or the like holding the necessary reagents, and usually including written instructions for the performance of assays. The kit may include containers to hold the materials during storage, use or both. The kit may include any configurations and compositions for performing the various assay formats described herein.

For example, a kit for detecting an ataxia-telangiectasia (A-T) gene mutation in a patient may contain an immobilizable or immobilized “capture” antibody which reacts with a phosphorylation form of an ATM kinase target for detecting the phosphorylation level of the ATM kinase target. The capture antibody may be labeled with a detectable label. The kit may further comprise a detectably labeled second (“detection”) antibody which binds to the capture antibody. Any conventional tag or detectable label may be part of the kit, such as a radioisotope, an enzyme, a chromophore or a fluorophore. The kit may also contain a reagent capable of precipitating immune complexes.

A kit according to the present disclosure can additionally include ancillary chemicals such as the buffers and components of the solution in which binding of antigen and antibody takes place.

### EXAMPLES

### Example 1

### Blood Processing

After informed consent, blood was collected from 7 normal daily controls, 16 healthy volunteers (unknowns), 10 obligate heterozygotes, and 6 unrelated A-T patients, into 10 mL tubes containing sodium heparin (Becton Dickinson Vacutainer Systems). Peripheral blood mononuclear cells (PBMCs) were isolated by centrifugation over a Ficoll-Hypaque density gradient (Amersham Pharmacia Biosciences). Mononuclear cells were transformed with Epstein-Barr virus and maintained at 37° C. and 5% CO₂ in RPMI 1640 (Gibco Invitrogen) containing 15% heat-inactivated fetal bovine serum (Hyclone) and 1% penicillin/streptomycin (Gibco Invitrogen). ATM mutations for the A-T patients studied are listed in Table 1.

TABLE 1
ATM mutations for the A-T patients studied

| ATLA# | Cell Type | Mutation A | Mutation B |
|---|---|---|---|
| AT203LA | LCL | 901G > A (Splicing Type I) | IVS28-159A > G (Splicing type II) |
| AT153LA | LCL | 8977C > T (Nonsense) | 8977C > T (Nonsense) |
| AT227LA | LCL | ND (not determined) | ND |
| L3 | LCL | 103C > T (Nonsense) | 103C > T (Nonsense) |
| AT7LA | LCL | 1563delAG (Frameshift) | 1563delAG (Frameshift) |
| AT187LA | LCL | 5908C > T (Nonsense) | 5908C > T (Nonsense) |
| AT228LA | LCL | 8395del10 (Frameshift) | 8395del10 (Frameshift) |
| AT224LA | LCL | 170G > A | 1402delAA |
| GRAT1 | LCL | 1215delT | 8756G > A |
| AT46LA | LCL | 5920delC (Frameshift) | ND |
| AT171LA | PBL | 6679C > T (R2227C) | 8633T > G (I2877R) |
| AT223LA | PBL | IVS23-2A > G (Splicing Type IV) | ND |
| AT2LA | PBL | 8494C > T (R2832C) | IVS19-22delAAT (Splicing Type V) |
| AT160LA | PBL | IVS45-IVS65 deletion | 7792C > T (R2597X) |
| | | (large deletion) | |
| AT27LA | PBL | 5515C > T (Q1839X) | 5712insA (Frameshift) |
| AT226LA | PBL | 1158delG (Frameshift) | 5228C > T (T1743I) |

### Example 2

### FC-pSMC1 Assay

PBMCs or LCLs were suspended in PBS and split into two aliquots. To produce DNA damage, the cells in one aliquot were irradiated (10 Gy) or treated with 1.5 μg/mL bleomycin. The cells were then incubated at 37° C. in 5% CO₂ for 1 hour, at which time they were fixed and permeabilized using Fix&Perm cell permeabilization kit (Caltag Laboratories; Invitrogen). Briefly, 100 μL fixation reagent A was used to resuspend, vortex-mix, and hold the cells for 3 minutes at room temperature, followed by the addition of 3 mL cold methanol. The methanol was added during vortex-mixing. The cells were incubated at 4° C. for 10 minutes and centrifuged at 300 g for 5 minutes. After centrifugation, the supernatants were removed and the cells were washed with 3 mL PBS plus 0.1% sodium azide and 5% fetal bovine serum, followed by centrifugation for 5 minutes at 300 g. We resuspended the cells in 100 μL permeabilization reagent B, added 5 μg rabbit anti-SMC1pSer966 antibody (it is an antibody specific to a phosphorylated form of SMC1 protein in which serine 966 is phosphorylated, NB 100-206; Novus), and incubated the preparation for 50 min at room temperature. After incubation, 3 mL wash buffer was added and the cells were centrifuged for 5 min at 300 g. The supernatant was removed and the cells were resuspended in 100 μL PBS containing 3 μg anti-rabbit-Ig fluorescein isothiocyanate-conjugated antibody (Jackson Immunoresearch Laboratories). Then the cells were incubated in the dark for 45 min at 20° C. The cells were washed with 3 mL wash buffer, centrifuged for 5 min at 300 g, resuspended in PBS, and fixed with 2% paraformaldehyde.

The samples were analyzed using a FACScaliber (BD Biosciences) with Cell Quest software, which plots geometric mean fluorescence intensity (GMFI) on the x axis using a log scale. GMFI peaks were converted to a linear scale for calculating % of daily control (% DC). The mean GMFI peak (linear scale) of untreated cells was subtracted from the GM peak FI of treated cells to yield the difference (δ GMFI), and the δ GMFIs for all samples were normalized against the δ GMFI of a healthy daily control (DC) and expressed as a proportion (% DC).

### Example 3

### Detection of ATM Kinase Activity by SMC1 Phosphorylation, Using LCLs

LCLs were suspended in PBS and split into two aliquots. To produce DNA damage, the cells in one aliquot were irradiated with 10 Gy.

### 1. Detection of ATM Kinase Activity by Immunoblotting

Nuclear extracts from 5-10 million LCLs were prepared following the manufacturer's protocol (NE-PER Nuclear and Cytoplasmic Extraction Reagents, Pierce, Rockford, Ill.). Nuclear lysate (25 μg) was electrophoresed on a 7.5% SDS polyacrylamide gel (PAGE), transferred onto polyvinylidene difluoride (PVDF) membrane (Bio-Rad, Hercules, Calif.), blocked with 5% milk, and incubated with a 1:1000 dilution of rabbit anti-SMC1pSER966, rabbit anti-SMC1, or rabbit anti-ATM (Novus Littleton, Colo.) overnight at 4° C. Horseradish peroxidase-conjugated Ig anti-rabbit antibody was added at a dilution of 1:3000 and incubated at room temperature for 40 minutes. All proteins were detected using an enhanced chemiluminescence kit (Amersham Pharmacia, Piscataway, N.J.).

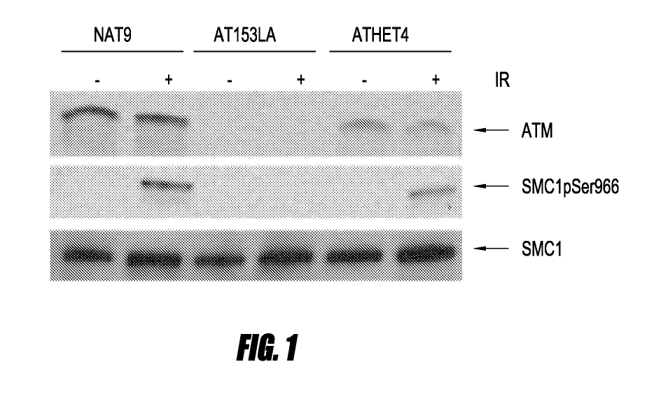

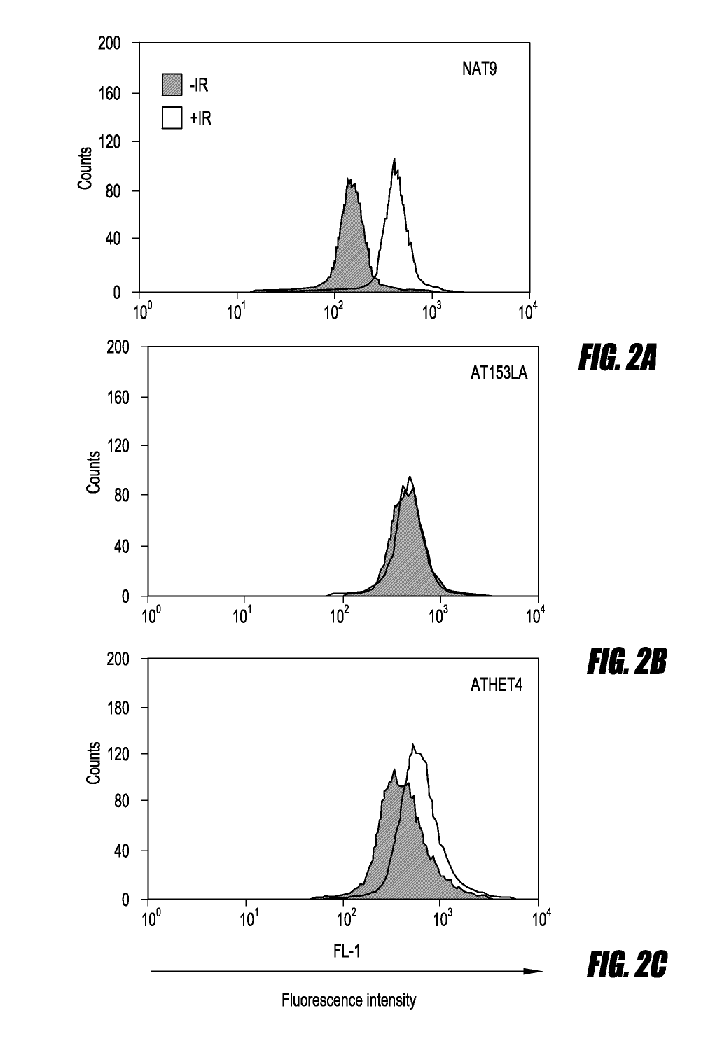

FIG. 1 demonstrates by immunoblotting that the phosphorylation of SMC1 on serine 966 after ionizing radiation (IR) with a 10 Gray (Gy) dose is absent in ATM-deficient cells (AT153LA) and reduced in an A-T heterozygote cell (ATHET4), compared with wild-type (WT) cells (NAT9). In addition, ATM protein levels are absent in A-T cells and reduced in A-T heterozygotes (FIG. 1). This indicated that measurement of phosphorylation levels is useful to determine A-T homozygotes and heterozygotes.

### 2. Detection of ATM Kinase Activity by Flow Cytometry

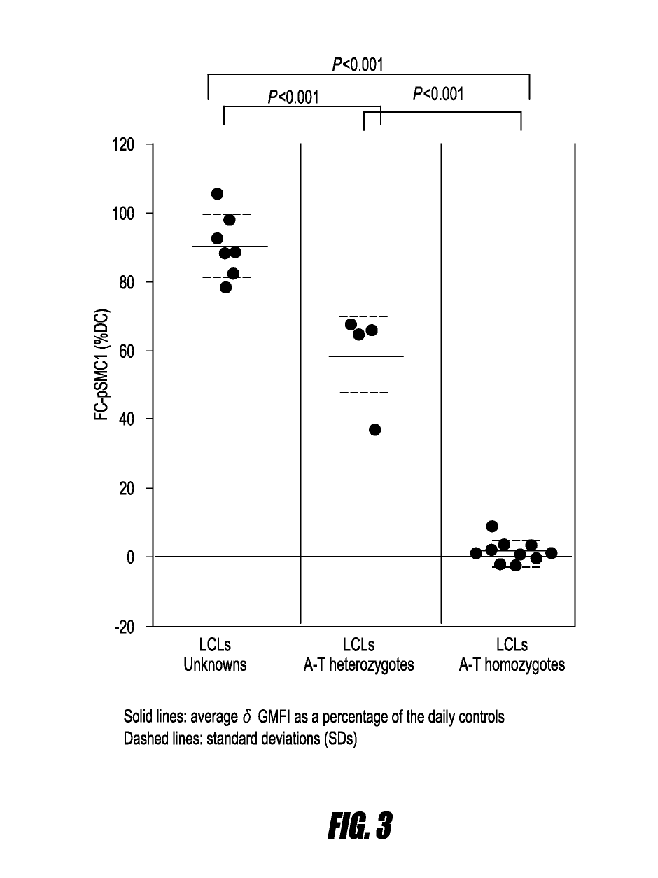

The LCLs pre- and post-10 Gy IR treatment were analyzed by the FC-pSMC1 assay. As shown in FIG. 2, a change in geometric mean fluorescence intensity (GMFI) was observed in NAT9 (wild type) when pre- and post-IR cells were compared. No change was observed after IR in ATM-deficient LCLs (AT153LA); and a reduced change (compared with WT) was observed in A-T heterozygotes LCLs (ATHET4).

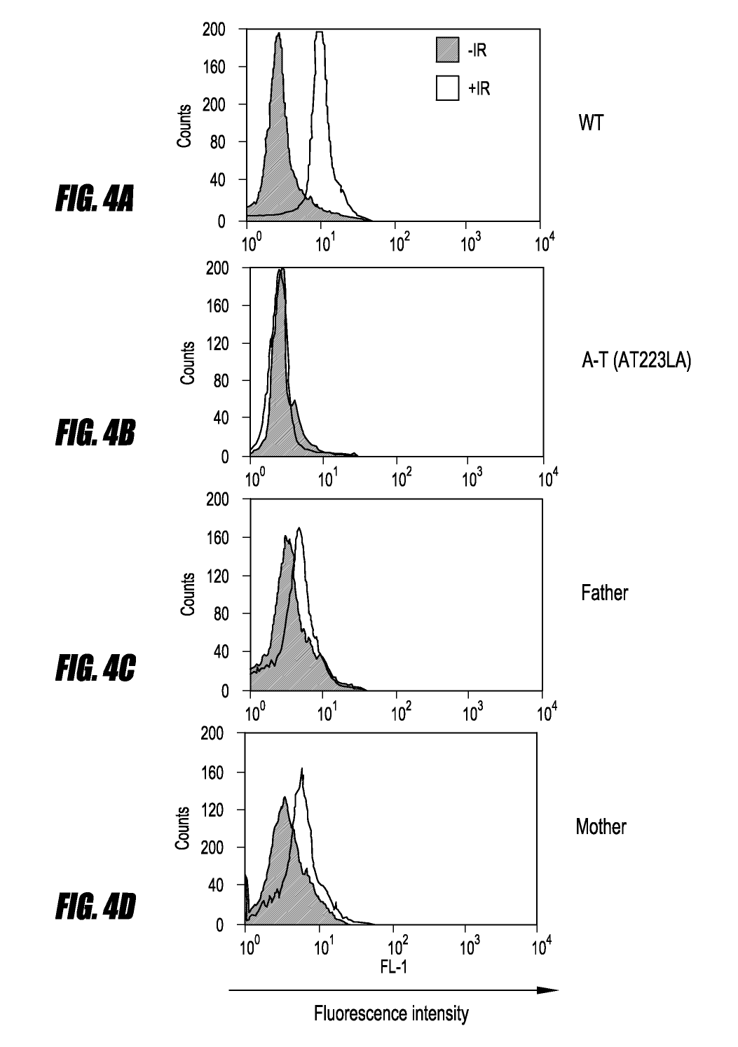

The aforementioned experiments were extended to LCLs from 7 healthy unknowns (WT), 4 A-T heterozygotes, and 10 A-T homozygous LCLs. The observed average IR-induced response, i.e., the change in the phosphorylation level of SMC1p966 in response to IR, was: 89.9% DC (standard deviation (SD): 9.2% DC) for unknown, 58.1% DC (14.4% DC) for A-T heterozygotes, and 0.83% DC (3.3% DC) (i.e., no change) for A-T homozygotes (FIG. 3). The δ GMFIs between genotypes were significantly different from each other (P=5×10⁻⁸, Kruskal-Wallis test). No change was observed using an unrelated antibody to aprataxin, another nuclear protein.

These results demonstrate that when compared to a standard reference change in the phosphorylation level of SMC1 in wildtype cells, a reduced change in the phosphorylation level of SMC1 in post-IR cells indicates the presence of an A-T gene mutation in the patient and that a lack of increase in the phosphorylation level of SMC1 in post-IR cells compared to pre-IR cells indicates the patient has ataxia-telangiectasia.

### Example 4

### Detection of ATM Kinase Activity by SMC1 Phosphorylation, Using PBMCs

Peripheral blood mononuclear cells (PBMCs) were suspended in PBS and split into two aliquots. To produce DNA damage, PBMCs in one aliquot were irradiated with various IR doses: 5, 10, or 20 Gy. A 10 Gy IR dose was found to optimize the increases in 8 GMFI. Dilutions of both the primary and secondary antibodies were also further optimized. After being irradiated with 10 Gy, the cells were subsequently incubated at 37° C. in 5% CO₂ for one hour, at which time, the cells were fixed and permeabilized as described in Example 2. And samples were analyzed using a FACScaliber (BD Biosciences) with Cell Quest software as described in Example 2.

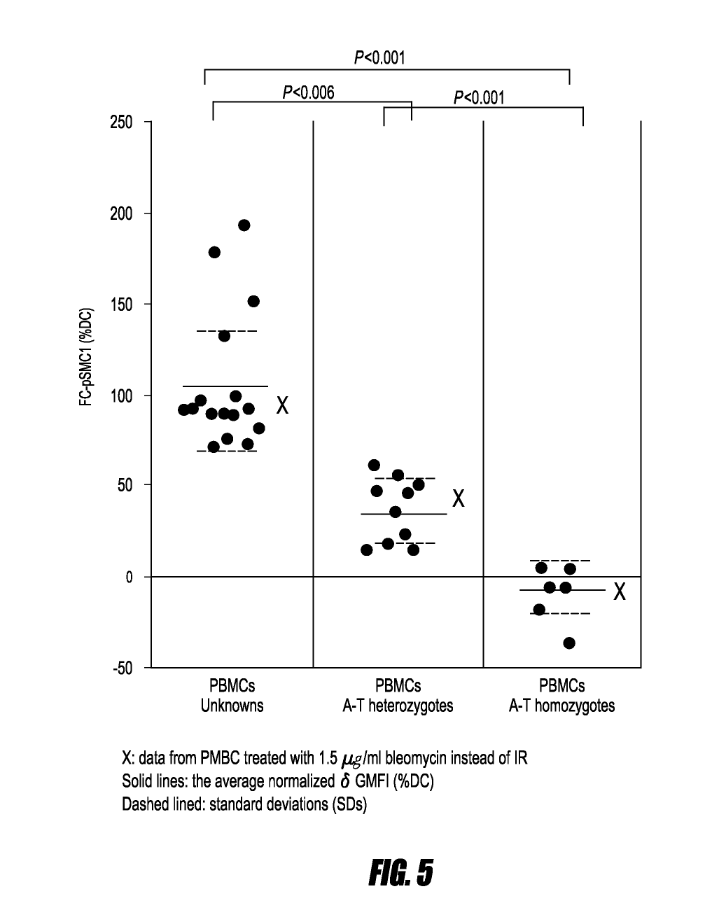

As shown in FIG. 4, the change in the phosphorylation level of SMC1p966 in response to IR for both parents was less than that of WT cells but clearly more than the negligible change seen with cells from the affected child (AT223LA). This was concordant with the previous immunoblot studies showing reduced amounts of ATM kinase activity in A-T heterozygotes (FIG. 1; Chun et al. (2003) Mol Genet Metab 80(4):437-443).

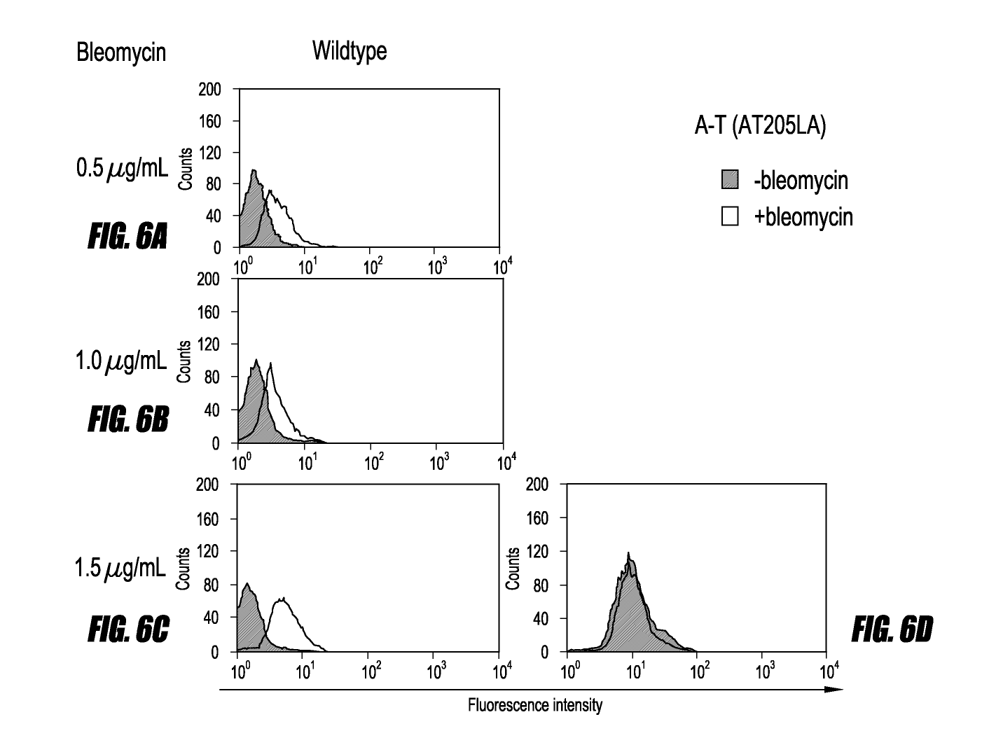

PMBCs from 16 healthy unknowns, 10 obligate A-T heterozygotes, and 6 unrelated homozygotes were tested next. Shown in FIG. 5, after 10 Gy IR, an average IR-induced response of 106.1% DC (37.6% DC), i.e., the standard reference change in the phosphorylation level of SMC1 in response to IR, was observed for 16 healthy unknowns. By comparison, for the fresh PBMCs isolated from the 10 obligate ATM heterozygotes, the average response to IR damage was significantly lower than that of healthy unknowns: 37.0% DC (18.7% DC) vs. 106.1% DC (37.6% DC) (P<0.006). In addition, responses of both the healthy unknowns and A-T heterozygotes were significantly larger than those of A-T homozygotes: 106.1% DC (37.6% DC) vs. −8.7% DC (16.2% DC), (P<0.001); and 37.0% DC (18.7% DC) vs. −8.7% DC (16.2% DC), (P<0.001). None of the responses for A-T homozygous PBMCs fell within the ranges for unknowns or obligate A-T heterozygotes (FIG. 5). The responses between genotypes were significantly different from each other (P<0.001, Kruskal-Wallis test). Further, the flow cytometry (FC)-pSMC1 assay data for A-T cells did not appear to be influenced by ATM mutations (Table 1).

In conclusion, FC-pSMC1 assay is a sufficiently sensitive and reliable test for identifying individuals with functionally compromised ATM kinase activity and distinguishing obligate A-T heterozygotes (i.e., parents of A-T patients) from WT and A-T homozygotes. Further, these results again demonstrate that when compared to a standard reference change in the phosphorylation level of SMC1 in wildtype cells, a reduced change in the phosphorylation level of SMC1 in post-IR cells indicates the presence of an A-T gene mutation in the patient, and that a lack of increase in the phosphorylation level of SMC1 in post-IR cells compared to pre-IR cells indicates the patient has ataxia-telangiectasia.

### Example 5

### Bleomycin as a Substitute DNA Damage-Inducing Agent for Irradiation

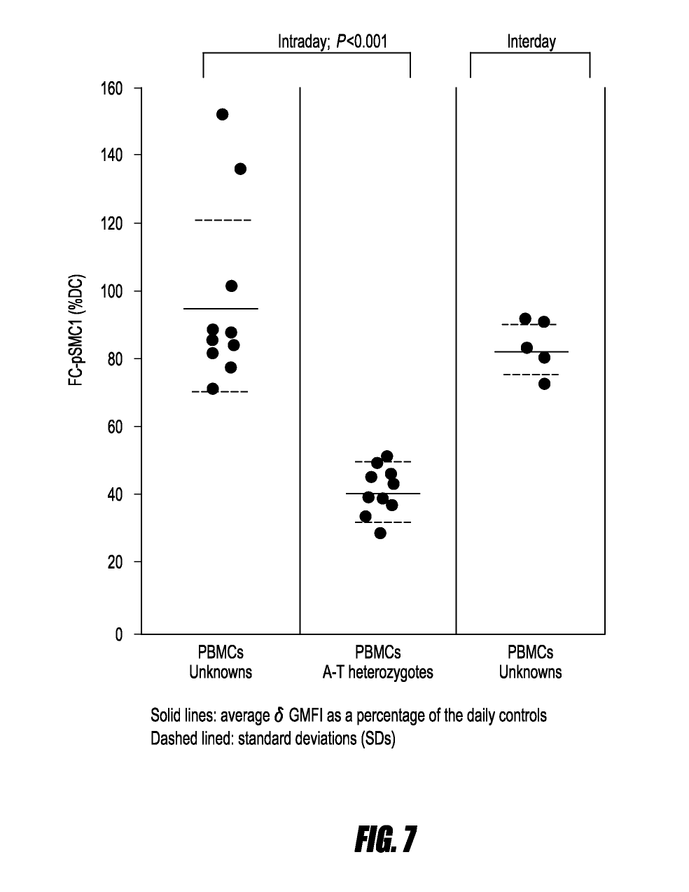

Because some clinical laboratories may not have access to a cell irradiator, bleomycin (a chemical inducer of double strand DNA breaks) was substituted for irradiation in the FC-pSMC1 assay (Povirk (1996) Mutat. Res 355:71-89). To optimize bleomycin dosage conditions, WT PBMCs were treated for 1 hour at 37° C. with 3 different doses of bleomycin: 0.5 μg/mL, 1.0 μg/mL and 1.5 μg/mL, which caused increases in delta FI (FIG. 6). A δ FI of 3.66 was observed with 1.5 μg/ml of bleomycin, which was approximately comparable to the δ FI seen with 10 Gy of ionizing radiation. Also, A-T PBMCs exhibited no increase in FI after damage with 1.5 μg/mL bleomycin (FIG. 6).

After optimizing bleomycin dosage conditions, PBMCs from a normal control, a second healthy unknown (i.e., 2 healthy individuals, 1 pre-designated to be the daily control; the other considered as a healthy unknown), an A-T heterozygote, and an A-T homozygote were treated with 1.5 μg/mL bleomycin for 1 hour at 37° C. The bleomycin treatment caused a change in GMFI (i.e., the δ GMFI) that was comparable to those seen in PBMCs treated with IR: unknown, 96.7% DC; A-T heterozygote, 48.6% DC; and A-T homozygote, −9.2% DC (i.e., no change) (FIG. 5). Because testing for most rare diseases is performed at a distant referral laboratory, blood samples are typically 1 to 3 days old when they are tested. However, no discernible pattern of change in % DC values was detected for 2- or 3-day-old shipped samples using either IR or bleomycin.

### Example 6

### Precision Studies

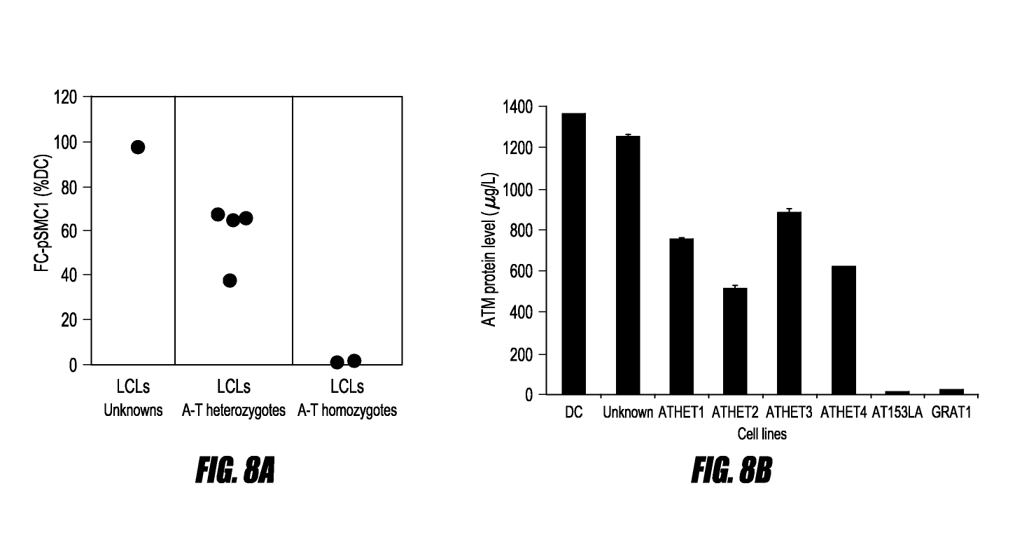

To determine intraday assay (within-run) variance (CV), PBMCs from a healthy daily control, a healthy unknown, and an A-T heterozygote donor were isolated and assayed 10 times in the same day by FC-pSMC1. The intraday assay variance (CV) for unknown was ≦27.5% DC, and ≦17.4% DC for A-T heterozygote (Table 2). The average increases in δ GMFIs as a percentage of the daily control for intraday sample variation were: 96.2% DC (26.5% DC) for the healthy unknown, 40.8% DC (7.1% DC) for the A-T heterozygote, (P<0.001) (FIG. 7, Table 2). For interday assay (between-run), PBMCs were collected from the same health donor in the intraday studies on 5 consecutive days and each sample was assayed singly each day. The interday assay variability (CV) for the healthy unknown was ≦9.4% DC (Table 2). The average increase in δ GMFI for the healthy unknown was 83.7% DC (7.9% DC) (FIG. 7, Table 2). Further, it was found that the assay could be performed using only 2 mL whole blood, allowing this assay to be used on very young children.

TABLE 2
Precision of FC-pSMC1 assay

| | | n | Mean (SD) | CV (%) |
|---|---|---|---|---|
| Intra-day^{a} | WT | 10 | 96.2 (26.5) | 27.5 |
| | A-THet | 10 | 40.8 (7.1) | 17.4 |
| Inter-day^{b} | WT | 5 | 83.7 (7.9) | 9.4 |

^{a}PBMCs were assayed a total of 10 times in the same day.
^{b}PBMCs were assayed one time on 5 different days.

### Example 7

### ATM-ELISA Assay

The ATM-ELISA assay that can be used to confirm a diagnosis of A-T on small numbers of PBMCs has been described previously (Butch et al. (2004) Clin Chem 50(12):2302-2308; US Patent Publication No. 2004/0029198). Nuclear lysates from LCLs and PBMCs were prepared by use of NE-PERTM Nuclear and Cytoplasmic Extraction Reagents (Pierce) according to the manufacturer's instructions. Protein concentrations of the nuclear lysates were measured using a modified Bradford method (Bio-Rad Laboratories), and 100 μg nuclear lysate was used for ATM protein quantification by immunoassay. Flat-bottomed, 96-well, high-binding enzyme immunoassay/RIA plates (Corning) were incubated with a purified mouse monoclonal antibody, ATM-2C1 (GeneTex), at 10 mg/L in PBS (pH 7.4) in a final volume of 120 μL for 6 hours at room temperature. After washing, the plate was blocked with PBS containing 30 g/L BSA and 1 mL/L Tween 20 for 45 minutes. Purified ATM protein (serial 2-fold dilutions starting at 640 μg/L) was added in triplicate and unknown nuclearcell/whole-cell lysates were added in duplicate. Calibrators and unknown samples were added in a total volume of 120 μL, with PBS containing 10 g/L BSA and 1 mL/L Tween 20 used as diluent. The plate was incubated overnight at room temperature, washed, and blocked; rabbit anti-ATM affinity-purified antibody (400-fold dilution in 120 μL volume; Novus Biologicals) was then added and incubated for 3 hours at room temperature. After washing, the plate was incubated with horseradish peroxidase-conjugated goat anti-rabbit IgG antibody (4000-fold dilution; Jackson ImmunoResearch Laboratories) for 3 hours at room temperature. After the plate was washed, 100 μL tetramethylbenzidine target (1-step Turbo TMB-ELISA; Pierce Biotechnology) was added to each well and incubated for 25 minutes; sulfuric acid (1 mol/L) was added to stop color formation and produce a yellow color. The absorbance of each well was measured at a wavelength of 450 nm and subtracted background absorbance at 630 nm. A calibration curve was generated using a linear curve-fitting program with a log-log scale (Microplate Manager Program; Bio-Rad), and ATM concentrations of unknown samples were determined from the calibration curve.

### Example 8

### Comparison of ATM-ELISA and FC-pSMC1

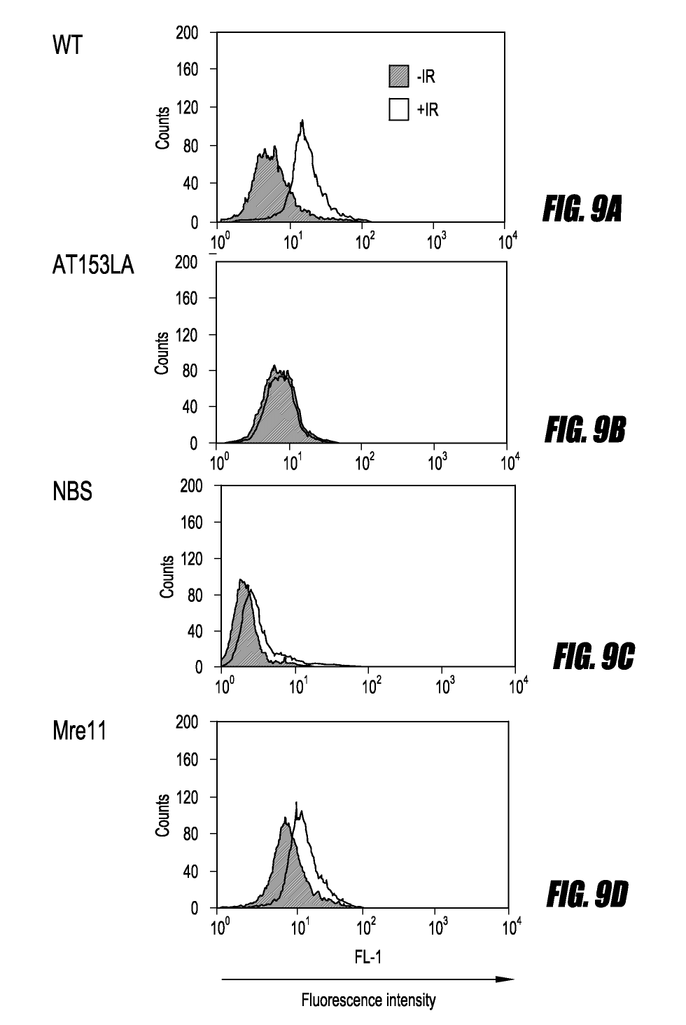

To determine whether the results from the FC-pSMC1 assay would be comparable to those seen when using the ATM-ELISA assay, ATM-ELISA was performed using 100 μg nuclear lysates isolated from LCLs of 1 healthy unknown, 4 obligate A-T heterozygotes (ATHET 1-4), and 2 A-T patients (AT153LA, GRAT1), as well as a daily control LCL (FIG. 8B). On average, ATM protein levels (also calculated as a percentage of the daily control LCL) were: 92.0% for the healthy unknown; 50.9% for A-T heterozygotes; and 1.1% for A-T homozygotes (FIG. 8B). These results were comparable to those seen using FC-pSMC1 (FIG. 8A): 97.3% DC (3.81% DC) for the healthy unknown, 58.0% DC (14.4% DC) for A-T heterozygotes, and 0.32% DC (0.35% DC) for A-T homozygotes. Therefore, the FC-pSMC1 and ATM-ELISA assays could be used adjunctively to identify the A-T homozygosity and heterozygosity of LCLs.

### Example 9

### Potential False-Positives for Other Genomic Instability Disorders

The FC-pSMCI assay was evaluated for potential false positive results for related genomic instability disorders that involve ATM activation and transphosphorylation, such as other radiosensitive disorders (Nijmegen breakage syndrome, Mre11 deficiency, DNA ligase IV deficiency, Fanconi anemia) or other early-onset ataxias (Mre11 deficiency, ataxia-oculomotor apraxia types 1 and 2).

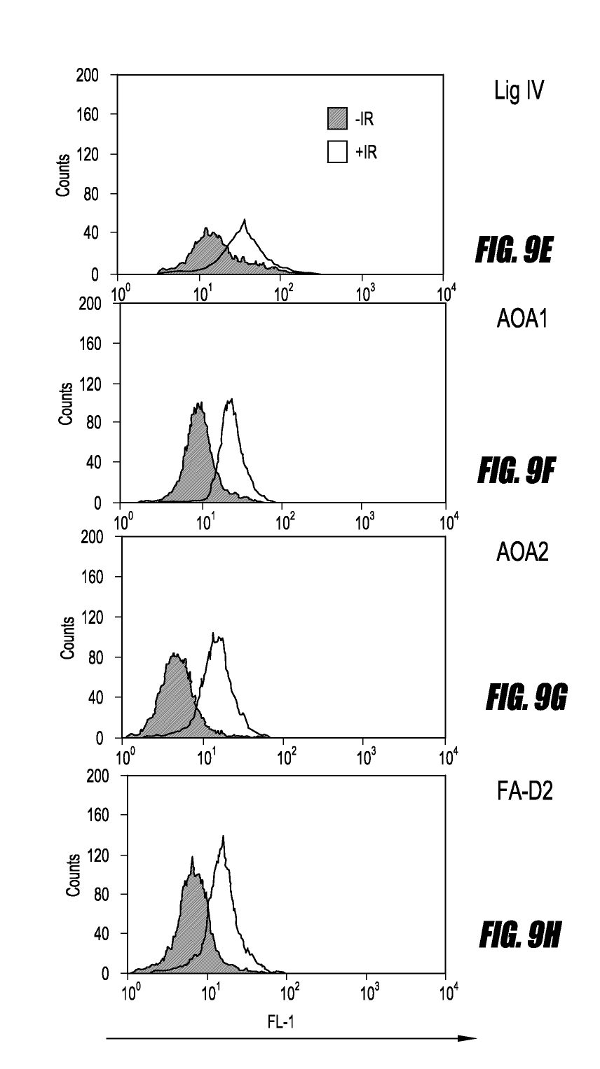

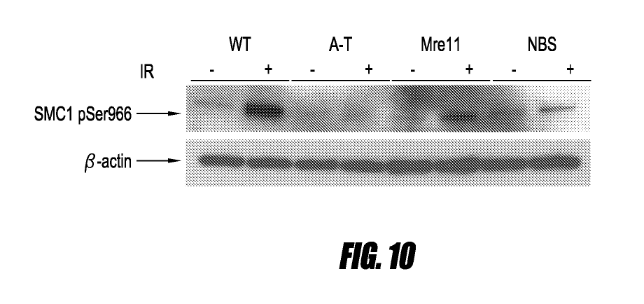

LCLs were extracted and prepared from patients with other radiosensitive disorders (Nijmegen breakage syndrome, Mre11 deficiency, DNA ligase IV deficiency, Fanconi anemia) or other early-onset ataxias (Mre11 deficiency, ataxia-oculomotor apraxia types 1 and 2). Only LCLs cells deficient in ATM, nibrin (NBS1), or Mre11 (ATLD) protein showed IR-response patterns different from that of WT (FIG. 9A-C). This might be explained by the finding that both nibrin and Mre11 proteins play prominent roles in recruiting ATM to DNA sites of double strand breaks. The nibrin (NBS)- and Mre11 (ATLD)-deficient cells showed changes in phosphorylation level of SMC1p966 in response to IR comparable to that of A-T heterozygotes; however, neither resembled the IR-response pattern of an A-T homozygote. These findings would not present a clinical testing problem for identifying A-T heterozygous patients since Mre11 and NBS patients have phenotypes that are easily distinguishable from the normal phenotype of A-T heterozygotes. Cells from patients with DNA Ligase IV deficiency, AOAI (aprataxin deficiency), AOA2 (senataxin deficiency), and FA-D2 (FANC-D2 deficiency) showed WT patterns (FIG. 9E-H). An immunoblot (FIG. 10) using nuclear extracts from some of the same LCLs gave comparable SMC1-S966 phosphorylation results. In conclusion, FC-pSMC1 assay is a useful and reliable test for distinguishing A-T patients from patients suffering from other genomic instability disorders.

### Example 10

### Screening a Patient for Susceptibility to Breast Cancer

A new patient suspected of being susceptible to breast cancer is identified. Nuclear cell lysates derived from the new patient's cells are tested to measure the ATM kinase activity based on the change in the phosphorylation level of an ATM kinase target in response to a DNA damage-inducing agent. The change in the phosphorylation level of the ATM kinase target in response to the DNA damage-inducing agent is determined by comparing the phosphorylation level of the ATM kinase target after the cells are treated with a DNA damage-inducing agent to the phosphorylation level of the ATM kinase target before the cells are treated with the DNA damage-inducing agent. The diagnostic tool described above is then used to determine whether the patient is at an increased risk of developing breast cancer based on the results of the ATM-kinase activity assay.

This information is combined with other factors known or suspected to be related to an individual's susceptibility to breast cancer (including family history, age, diet, status as a smoker, ethnicity, geographic and/or environmental factors, etc.) to generate an overall prediction of the patient's susceptibility to breast cancer. This overall prediction information is then used for patient counseling, further testing, and/or medical treatment as deemed necessary. These steps allow the patient to have more information about her particularized risk for breast cancer and allow her to take actions which can lead to a healthier and longer life.

This procedure is performed on individuals believed to be at increased risk for breast cancer. This increased risk can be based on family history of breast cancer, family history of A-T or A-T carriers, or on other factors known or suspected to be related to breast cancer. Alternatively, the procedure can be performed on any individual to assist in calculating the individual's risk of developing breast cancer, or of having children who may develop breast cancer.

Further, the procedure can be used to assess risks of developing other conditions that are found to be related to levels of functional ATM protein. These other conditions can include various forms of cancer, neurological disorders, and heart disease, particularly ischemic heart disease. Any other condition that is actually or theoretically correlated to the A-T gene and/or the ATM protein may also be considered.

The techniques disclosed above can be used in a variety of contexts. Some embodiments include screening or testing for susceptibility to various disorders, including cancer or cardiovascular disease. Such screening or testing may be performed, for example, before a patient undergoes radiation therapy, chemotherapy, treatment with a radiomimetic or radioprotective agent, or prior to employment in a workplace involving increased risk of exposure to carcinogens or radiation (such as airline personnel, uranium mine workers, X-ray lab technicians, outer space flight, etc.) Some embodiments include diagnosing patients with DNA repair/genomic instability disorders.

Unless defined otherwise, all technical and scientific terms used herein have the same meaning as commonly understood by one of ordinary skill in the art to which this invention belongs. Although the invention has been described with reference to embodiments and examples, it should be understood that various modifications can be made without departing from the spirit of the invention. All references cited herein are expressly incorporated herein by reference in their entirety.

1. A method of detecting an ataxia-telangiectasia (A-T) gene mutation in a patient comprising:
measuring the phosphorylation level of an ATM kinase target in a biological sample from the patient;
contacting the biological sample with a DNA damage-inducing agent;
measuring the phosphorylation level of the ATM kinase target in the biological sample after treatment with the DNA damage-inducing agent; and
comparing the measured phosphorylation level before and after treatment with the DNA damage-inducing agent to determine the presence of an A-T gene mutation in the patient.

2. (canceled)

3. (canceled)

4. (canceled)

5. The method of claim 1 wherein the ATM kinase target is SMC1.

6. The method of claim 1 wherein the DNA damage-inducing agent is ionizing radiation (IR) or bleomycin.

7. (canceled)

8. The method of claim 1, wherein the biological sample is blood of an amount no greater than 3 mL.

9. The method of claim 1, wherein the biological sample comprises peripheral blood mononuclear cells.

10. The method of claim 1, wherein the biological sample comprises lymphoblastoid cells.

11. The method of claim 1, wherein measuring the phosphorylation level of the ATM kinase target comprises flow cytometry or immunoblot analysis.

12. (canceled)

13. A method of screening for susceptibility of a disorder in a patient comprising:
measuring the phosphorylation level of an ATM kinase target in a biological sample from the patient;
contacting the biological sample with a DNA damage-inducing agent;
measuring the phosphorylation level of the ATM kinase target in the biological sample after treatment with the DNA damage-inducing agent; and
comparing the measured phosphorylation level before and after treatment with the DNA damage-inducing agent to determine the susceptibility of a disorder in the patient.

14. The method of claim 12 wherein the disorder is ataxia-telangiectasia, cancer, breast cancer, neurological disorder, or heart disease.

15. (canceled)

16. (canceled)

17. (canceled)

18. (canceled)

19. The method of claim 12 wherein the ATM kinase target is SMC1.

20. The method of claim 12, wherein the DNA damage-inducing agent is ionizing radiation (IR) or bleomycin.

21. (canceled)

22. The method of claim 12 wherein the biological sample comprises peripheral blood mononuclear cells.

23. The method of claim 12 wherein the biological sample comprises lymphoblastoid cells.

24. The method of claim 12, wherein measuring the phosphorylation level of the ATM kinase target comprises flow cytometry.

25. The method of claim 12, measuring the phosphorylation level of the ATM kinase target comprises immunoblot analysis.

26. A kit for detecting an ataxia-telangiectasia (A-T) gene mutation in a patient, comprising:
a DNA damage-inducing agent;
an antibody for detecting the phosphorylation level of an ATM kinase target; and
an instruction for contacting the DNA damage-inducing agent with a biological sample from the patient.

27. The kit of claim 26, wherein said antibody is labeled.

28. The kit of claim 27, wherein the second antibody is labeled with a fluorophore.

29. The kit of claim 26, further comprising a second antibody that binds to the antibody for detecting the phosphorylation level of the ATM kinase target.

30. The kit of claim 29, wherein the second antibody is labeled.

31. (canceled)
